(19) United States
(12) Patent Application Publication
(10) Pub. No.: US 2008/0026410 A1
(43) Pub. Date: Jan. 31, 2008

(54) Biomarkers for Bladder Cancer

(76) Inventor: Antonia Vlahou, Nolfolk, VA (US)

Correspondence Address: FOLEY AND LARDNER LLP;SUITE 500, 3000 K STREET NW, WASHINGTON, DC, 20007, US

(21) Appl. No.: 11/632,423

(22) PCT Filed: Jul. 20, 2005
(86) PCT No.: PCT/US05/25632

§ 371 Date: May 8, 2007

Related U.S. Application Data
(60) Provisional application No. 60/632,423, filed on Dec. 2, 2004.

(51) Int. Cl. G01N 33/574; C12M 3/00

(52) U.S. Cl. 435/7.23; 435/287.2

(57) ABSTRACT

The present invention provides protein-based biomarkers and biomarker combinations that are useful in qualifying bladder cancer status in a patient. In particular, the biomarkers of this invention are useful to classify a subject sample as bladder cancer or non-bladder cancer. The biomarkers can be detected by SELDI mass spectrometry.

This invention was made with government support under DA85067 awarded by the National Institutes of Health. The government has certain rights in the invention. The invention also was supported by the Elsa U Pardee Research Foundation and the Virginia Prostate Center.

The present invention is supported by the Elsa U Pardee Research Foundation, the National Cancer Institute Early Detection Research Network (DA85067) and the Virginia Prostate Center. The Government may have certain rights in the invention.

### BACKGROUND OF THE INVENTION

Bladder cancer is the second most common genitourinary malignancy accounting for approximately 5% of all newly diagnosed cancers in the United States (Klein et al., Cancer 82 (2):49-354 (1998)). More than 90% are of the transitional cell carcinoma (TCC) histology (Stein et al., J. Urol. 160:645-659 (1998)). At present, the most reliable way of diagnosis and surveillance of bladder cancer is by cystoscopic examination and bladder biopsy for histologic confirmation. The invasive and labor-intensive nature of this procedure presents a challenge to develop better, less costly, and non-invasive diagnostic tools. Urine cytology has for many years been the ‘gold standard’ of the non-invasive approaches. It has high specificity and provides the advantage over biopsy of screening the entire urothelium (Klein et al., Cancer 82 (2): 49-354 (1998); Stein et al., J. Urol. 160:645-659 (1998)). However, its high false negative rate, particularly for low grade tumors, has limited its use as an adjunct to cystoscopy.

Many non-invasive molecular diagnostic tests have been developed based on an ever increasing knowledge about the molecular alterations associated with bladder cancer pathogenesis. The bladder tumor antigen (BTA) (Schamhart et al., Eur. Urol. 34: 99-106 (1998)), the BTA stat (Sarosdy et al., Urology 50:349-53 (1997)), the fibrinogen/fibrin degradation products (FDP) (Schmnetter et al., J. Urol. 158:801-805 (1997)) and the nuclear matrix protein-22 (NP-22) (Soloway et al., J. Urol. 156:363-367 (1996)) tests, have been approved by the FDA to be used in conjunction with cystoscopy. See Grossman et al., Urol. Oncology 5:3-10 (2000) for review. Additional molecular assays currently being evaluated for their diagnostic/prognostic utility are the Telomerase (Hoshi et al., Urol. Onc. 5:25-30 (2000)), Immunocyt (Fradet et al., Can. J. Urol. 1997, 4:400-5 (1997)) and hyaluronic acid/hyaluronidase (Pham et al., Cancer Research 57:778-783 (1997); Lokeshwar et al., Cancer Research 57:773-777 (1997)) tests, microsatellite analysis (Steiner et al., Nat. Aced. 6:621-624 (1997)), as well as assays detecting blood group antigens (Golijanin et al., Urology 46(2):173-177 (1995)), carcinoembryonic antigen (Liu et al., J. Urol. 137:1258 (1987)), p 53 and retinoblastoma proteins (Grossman et al., Urol. Oncology 5:3-10 (2000)), E cadherin (Banks et al., J. Clin. Pathol. 48:179-180 (1995); Protheroe et al., British J. Cancer 80(1/2):273-8 (1999)), and various growth factors (Halachmi et al., British J. Urology 82:647-654 (1998)).

The effectiveness of any diagnostic test depends on its specificity and selectivity, or the relative ratio of true positive, true negative, false positive and false negative diagnoses. Methods of increasing the percent of true positive and true negative diagnoses for any condition are desirable medical goals. In the case of bladder cancer, the present diagnostic tests are not completely satisfactory for the reasons described above.

One of the recent technological advances in facilitating protein profiling of complex biologic mixtures is the ProteinChip® surface-enhanced laser desorption/ionization time of flight mass spectrometry (SELDI-TOF-MS) (Kuwata, H., et al., Biochem. Biophys. Res. Commun. 245:764-773 (1998); Merchant, M. et al., Electrophoresis 21:1164-1177 (2000)). This technology utilizes protein chips coated with a chemical to affinity capture protein molecules from complex mixtures. The SELDI system is an extremely sensitive and rapid method that analyzes complex mixtures of proteins and peptides. Applications of this technology show great potential for the early detection of prostate, breast, esophageal, ovarian, and hepatic cancers (Paweletz, C., et al., Drug Dev. Res. 49:3442 (2000); Wright, G., et al., Prostate Cancer and Prostate Diseases 2:264-276 (1999); Cazares, L. H., et al., Clin. Cancer Res. 8:2541-2552 (2002); Paweletz, C., et al., Disease Markers 17:201-307 (2001)). Moreover, the analysis of SELDI data by “artificial intelligence” algorithms can lead to the identification of serum protein “fingerprints” of prostate, ovarian and breast cancers (Qu, Y., et al., Clin. Chem. 48(10):1835-43 (2002); Petricoin, E., et al., LANCET 359:572-577 (2002); Li, J., et al., Clin. Chem. 48(8):1296-304 (2002); Vlahou, A., et al., J. Biomed. and Biotechnol. 2003(5):308-314; Vlahou, A., et al., Clin. Breast Cancer 4(3):203-9; Vlahou, A., et al., American J. Pathology 158(4):1491-1502 (2001)).

The identification and simultaneous analysis of a panel of biomarkers, representative of the various biological characteristics of the cancer, has greater potential for improving the early detection/diagnosis of bladder cancer. Moreover, in an economy-conscious environment in which cost-effective medicine is an overriding concern, physicians treating cancer patients need convenient, efficient methods to rapidly diagnose bladder cancer and to evaluate responses to therapy. The present invention meets this and other goals.

### BRIEF SUMMARY OF THE INVENTION

The present invention provides a method for qualifying bladder cancer status in a subject, the method comprising: (a) measuring at least one biomarker in a biological sample from the subject, wherein the at least one biomarker is selected from the group consisting of Marker 1, Marker 2, Marker 3, Marker 4, Marker 5, Marker 6, Marker 7, Marker 8, Marker 9, Marker 10, Marker 11, and Marker 12; and (b) correlating the measurement with bladder cancer status. The biological sample can be any suitable sample, such as urine or serum.

In one embodiment, a plurality of biomarkers is measured. The plurality may comprise at least 3 biomarkers or at least 4 biomarkers.

In another embodiment, one or more biomarkers is also measured in the subject: Marker 13, Marker 14, Marker 15, Marker 16, Marker 17, and Marker 18.

The invention further provides a method for qualifying bladder cancer status in a subject comprising: (a) measuring a plurality of biomarkers in a biological sample from the subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and (b) correlating the measurement with bladder cancer status. The biological sample can be any suitable sample, such as urine or serum.

In another embodiment, the methods for qualifying bladder cancer status comprise measuring the biomarkers by capturing the biomarker on an adsorbent surface of a SELDI probe and detecting the captured biomarkers by laser desorption-ionization mass spectrometry. Any adsorbent surface can be used to capture the biomarkers. For example, the adsorbent on the substrate can be a cation exchange adsorbent, a biospecific adsorbent, etc.

In another embodiment, the methods for qualifying bladder cancer status comprise measuring the biomarkers by immunoassay.

In another embodiment, the bladder cancer status is selected from bladder cancer and non-bladder cancer.

In another embodiment, the correlation is performed by a software classification algorithm.

In another embodiment, the methods for qualifying bladder cancer status comprise the additional steps of: (c) managing subject treatment based on the status and (d) measuring the at least one biomarker after subject management.

The invention further provides a method for measuring at least one biomarker in a sample from a subject, wherein the at least one biomarker is selected from the group consisting of Marker 1, Marker 2, Marker 3, Marker 4, Marker 5, Marker 6, Marker 7, Marker 8, Marker 9, Marker 10, Marker 11, and Marker 12. The biological sample can be any suitable sample, such as urine or serum.

In one embodiment, a plurality of biomarkers is measured. The plurality may comprise at least 3 biomarkers or at least 4 biomarkers.

In another embodiment, one or more biomarkers is also measured in the subject: Marker 13, Marker 14, Marker 15, Marker 16, Marker 17, and Marker 18.

The invention also provides a method comprising measuring a plurality of biomarkers in a sample from a subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18. The biological sample can be any suitable sample, such as urine or serum.

In another embodiment, the methods of measuring biomarkers comprise capturing the biomarker on an adsorbent surface of a SELDI probe and detecting the captured biomarkers by laser desorption-ionization mass spectrometry. Any adsorbent surface can be used to capture the biomarkers. For example, the adsorbent on the substrate can be a cation exchange adsorbent, a biospecific adsorbent, etc.

The invention also provides kits comprising: (a) a solid support comprising at least one capture reagent attached thereto, wherein the capture reagent binds at least one biomarker selected from the group consisting of Marker 1, Marker 2, Marker 3, Marker 4, Marker 5, Marker 6, Marker 7, Marker 8, Marker 9, Marker 10, Marker 11, and Marker 12; and (b) instructions for using the solid support to detect the at least one biomarker.

In another embodiment, the kits further comprise instructions for using the solid support to detect one or more of the following biomarkers: Marker 13, Marker 14, Marker 15, Marker 16, Marker 17, and Marker 18.

In some embodiments, the kits comprise instructions for using the solid support to detect a plurality of biomarkers. The plurality may comprise at least 3 biomarkers or at least 4 biomarkers.

The invention further provides kits comprising: (a) a solid support comprising at least one capture reagent attached thereto, wherein the capture reagent binds a plurality of biomarkers, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and (b) instructions for using the solid support to detect the plurality of biomarkers.

In one embodiment, the solid support comprising a capture reagent is a SELDI probe. In another embodiment, the capture reagent is a cation exchange adsorbent.

In another embodiment, the kits additionally comprise (c) an anion exchange chromatography adsorbent.

The invention also provides kits comprising: (a) a solid support comprising at least one capture reagent attached thereto, wherein the capture reagents bind at least one biomarker selected from the group consisting of Marker 1, Marker 2, Marker 3, Marker 4, Marker 5, Marker 6, Marker 7, Marker 8, Marker 9, Marker 10, Marker 11, and Marker 12; and (b) a container containing at least one of the biomarkers.

In one embodiment, the kits further comprise instructions for using the solid support to detect one or more of the following biomarkers: Marker 13, Marker 14, Marker 15, Marker 16, Marker 17, and Marker 18.

In some embodiments, the kits comprise a plurality of biomarkers. The plurality may comprise at least 3 biomarkers or at least 4 biomarkers.

The invention also provides kits comprising: (a) a solid support comprising at least one capture reagent attached thereto, wherein the capture reagents bind a plurality of biomarkers, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12, and at least one biomarker is selected from the group consisting of Marker 13, Marker 14, Marker 15, Marker 16, Marker 17, and Marker 18; and (b) a container containing at least one of the biomarkers.

In some embodiments, the container contains a plurality of biomarkers. The plurality may comprise at least 3 biomarkers or at least 4 biomarkers.

In one embodiment, the solid support comprising a capture reagent is a SELDI probe. In another embodiment, the capture reagent is a cation exchange adsorbent.

In another embodiment, the kits additionally comprise (c) an anion exchange chromatography adsorbent.

The invention further provides a software product comprising: (a) code that accesses data attributed to a sample, the data comprising measurement of at least one biomarker in the sample, the biomarker selected from the group consisting of Marker 1, Marker 2, Marker 3, Marker 4, Marker 5, Marker 6, Marker 7, Marker 8, Marker 9, Marker 10, Marker 11, and Marker 12; and (b) code that executes a classification algorithm that classifies the bladder cancer status of the sample as a function of the measurement.

In one embodiment, the classification algorithm classifies the bladder cancer status of the sample further as a function of the measurement of one or more of the following biomarkers: Marker 13, Marker 14, Marker 15, Marker 16, Marker 17, and Marker 18.

In some embodiments, the classification algorithm classifies the bladder cancer status of the sample as a function of the measurement of a plurality of biomarkers. The plurality may comprise at least 3 biomarkers or at least 4 biomarkers.

The invention further provides a software product comprising: (a) code that accesses data attributed to a sample, the data comprising measurement of a plurality of biomarkers in the sample, and wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and (b) code that executes a classification algorithm that classifies the bladder cancer status of the sample as a function of the measurement.

The invention further provides purified biomolecules selected from the group of biomarkers consisting of Marker 1, Marker 2, Marker 3, Marker 4, Marker 5, Marker 6, Marker 7, Marker 8, Marker 9, Marker 10, Marker 11, and Marker 12.

The invention further provides a method comprising detecting a biomarker from the ground consisting of Marker 1, Marker 2, Marker 3, Marker 4, Marker 5, Marker 6, Marker 7, Marker 8, Marker 9, Marker 10, Marker 11, and Marker 12 by mass spectrometry or immunoassay.

The invention further provides a method comprising detecting a plurality of biomarkers by mass spectrometry or immunoassay, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18.

The invention also provides a method comprising communicating to a subject a diagnosis relating to bladder cancer status determined from the correlation of biomarkers in a sample from the subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12.

The invention also provides a method comprising communicating to a subject a diagnosis relating to bladder cancer status determined from the correlation of a plurality of biomarkers in a sample from the subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18.

In one embodiment, the diagnosis is communicated to the subject via a computer-generated medium.

The invention also provides a method for identifying a compound that interacts with any of the biomarkers selected from the group consisting of Marker 1, Marker 2, Marker 3, Marker 4, Marker 5, Marker 6, Marker 7, Marker 8, Marker 9, Marker 10, Marker 11, and Marker 12, wherein the method comprises: (a) contacting the biomarker with a test compound; and (b) determining whether the test compound interacts with the biomarker.

The invention also provides a method for modulating the concentration of a biomarker selected from the group consisting of Marker 1, Marker 2, Marker 3, Marker 4, Marker 5, Marker 6, Marker 7, Marker 8, Marker 9, Marker 10, Marker 11, Marker 12, Marker 13, Marker 14, Marker 15, Marker 16, Marker 17, and Marker 18 in a cell, wherein the method comprises: (a) contacting the cell with a protease inhibitor, wherein the protease inhibitor prevents cleavage of the biomarker.

The invention further provides a method of treating a condition in a subject, wherein the method comprises administering to a subject a therapeutically effective amount of a compound which modulates the expression or activity of a protease which cleaves a biomarker selected from the group consisting of Marker 1, Marker 2, Marker 3, Marker 4, Marker 5, Marker 6, Marker 7, Marker 8, Marker 9, Marker 10, Marker 11, Marker 12, Marker 13, Marker 14, Marker 15, Marker 16, Marker 17, and Marker 18. In one embodiment, the condition is bladder cancer.

### BRIEF DESCRIPTION OF THE DRAWINGS

FIG. 1 shows a decision tree for classifying a sample as bladder cancer or non-bladder cancer using certain biomarkers of this invention, as utilized in Example 1. “C” represents bladder cancer patients and “B” represents “benign” patients (those that are normal or have benign or other cancers). The squares are the primary nodes and the circles indicate terminal nodes. The mass value in the root nodes (in kDa) are followed by the intensity cutoff levels of the splitter as well as the number of samples involved.

FIG. 2 depicts mass spectra of the peaks (arrows) forming the main splitters of the decision tree.

FIG. 3 depicts the intensity distribution of the peaks forming the main splitters of the decision tree. Each square corresponds to a decision node of the tree shown in FIG. 1. The mass of the main splitter (in kDa), its intensity value in the cancer (“C”) and non-cancer (normal and benign, or “B”) samples, and the intensity cut-off values that form the splitting rule are shown.

### DETAILED DESCRIPTION OF THE INVENTION

### I. Introduction

A biomarker is an organic biomolecule, the presence of which in a sample is used to determine the phenotypic status of the subject (e.g., bladder cancer patient v. normal or non-bladder cancer patient). In a preferred embodiment, the biomarker is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

### II. Biomarkers for Bladder Cancer

A. Biomarkers

This invention provides polypeptide-based biomarkers that are used to distinguish subjects with bladder cancer from subjects that are normal or with non-bladder cancer. The biomarkers are preferably differentially present in subjects having bladder cancer, versus subjects who are normal or have non-bladder cancer. The biomarkers are characterized by mass-to-charge ratio as determined by mass spectrometry, by the shape of their spectral peak in time-of-flight mass spectrometry and by their binding characteristics to adsorbent surfaces. These characteristics provide one method to determine whether a particular detected biomolecule is a biomarker of this invention. These characteristics represent inherent characteristics of the biomolecules and not process limitations in the manner in which the biomolecules are discriminated. In one aspect, this invention provides these biomarkers in isolated form.

The biomarkers were discovered using SELDI technology employing ProteinChip arrays from Ciphergen Biosystems, Inc. (Fremont, Calif.) (“Ciphergen”). Urine samples were collected from subjects diagnosed with bladder cancer and subjects diagnosed as normal. The samples were fractionated by anion exchange chromatography. Fractionated samples were applied to SELDI biochips and spectra of polypeptides in the samples were generated by time-of-flight mass spectrometry on a Ciphergen PBSII mass spectrometer. The spectra thus obtained were analyzed by Ciphergen Express™ Data Manager Software with Biomarker Wizard and Biomarker Pattern Software from Ciphergen Biosystems, Inc. The mass spectra for each group were subjected to scatter plot analysis. A Mann-Whitney test analysis was employed to compare bladder cancer and control groups for each protein cluster in the scatter plot, and proteins were selected that differed significantly (p<0.0001) between the two groups. This method is described in more detail in the Example Section.

The biomarkers thus discovered are presented in Tables 1 and 2. The “ProteinChip assay” column of Table 2 refers to the type of biochip to which the biomarker binds and the wash conditions, as per the Example.

TABLE 1

| Marker No. | Mass (Da) |
|---|---|
| 1 | M2670.00 |
| 2 | M4210.00 |
| 3 | M5400.00 |
| 4 | M5510.00 |
| 5 | M5580.00 |
| 6 | M5700.00 |
| 7 | M8490.00 |
| 8 | M9100.00 |
| 9 | M10800.00 |
| 10 | M17000.00 |
| 11 | M56500.00 |
| 12 | M67000.00 |
| 13 | M3380.00 |
| 14 | M3540.00 |
| 15 | M4960.00 |
| 16 | M5830.00 |
| 17 | M7070.00 |
| 18 | M9420.00 |

TABLE 2

| | | | Up or down | |
|---|---|---|---|---|
| | | | regulated in | |
| Marker | Mass | | bladder | |
| No. | (Da) | P-Value | cancer | ProteinChip ® assay |
| 1 | M2670.00 | 0.006 | down | WCX, wash with 100 mM |
| | | | | Na acetate pH 4 |
| 2 | M4210.00 | 0.042 | up | WCX, wash with 100 mM |
| | | | | Na acetate pH 4 |
| 4 | M5510.00 | 0.044 | both | WCX, wash with 100 mM |
| | | | | Na acetate pH 4 |
| 8 | M9100.00 | 0.25 | up | WCX, wash with 100 mM |
| | | | | Na acetate pH 4 |
| 14 | M3540.00 | 0.007 | down | WCX, wash with 100 mM |
| | | | | Na acetate pH 4 |
| 15 | M4960.00 | <0.0001 | down | WCX, wash with 100 mM |
| | | | | Na acetate pH 4 |
| 17 | M7070.00 | 0.41 | up | WCX, wash with 100 mM |
| | | | | Na acetate pH 4 |

The biomarkers of this invention are characterized by their mass-to-charge ratio as determined by mass spectrometry. The mass-to-charge ratio of each biomarker is provided in Table 1 after the “M.” Thus, for example, M2670.00 has a measured mass-to-charge ratio of 2670.00. The mass-to-charge ratios were determined from mass spectra generated on a Ciphergen Biosystems, Inc. PBS II mass spectrometer. This instrument has a mass accuracy of about +/−0.3 percent. Additionally, the instrument has a mass resolution of about 400 to 1000 m/dm, where m is mass and dm is the mass spectral peak width at 0.5 peak height. The mass-to-charge ratio of the biomarkers was determined using Biomarker Wizard™ software (Ciphergen Biosystems, Inc.). Biomarker Wizard assigns a mass-to-charge ratio to a biomarker by clustering the mass-to-charge ratios of the same peaks from all the spectra analyzed, as determined by the PBSII, taking the maximum and minimum mass-to-charge-ratio in the cluster, and dividing by two. Accordingly, the masses provided reflect these specifications.

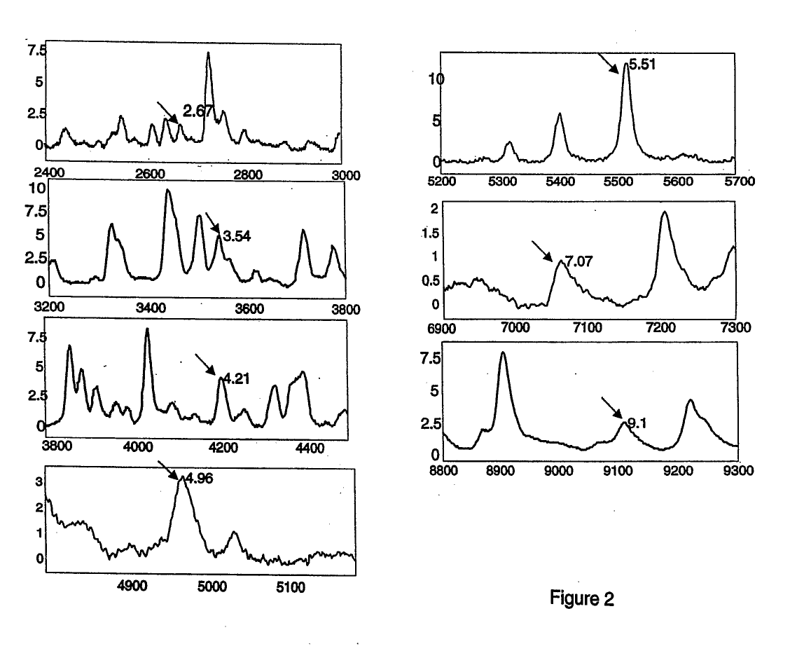

The biomarkers of this invention are further characterized by the shape of their spectral peak in time-of-flight mass spectrometry. Mass spectra showing peaks representing the biomarkers are presented in FIG. 2.

The biomarkers of this invention are further characterized by their binding properties on chromatographic surfaces. Most of the biomarkers bind to cation exchange adsorbents (e.g., the Ciphergen® WCX ProteinChip® array) after washing with 100 mM sodium acetate at pH 4.

Because the biomarkers of this invention are characterized by mass-to-charge ratio, binding properties and spectral shape, they can be detected by mass spectrometry without knowing their specific identity. However, if desired, biomarkers whose identity is not determined can be identified by, for example, determining the amino acid sequence of the polypeptides. For example, a biomarker can be peptide-mapped with a number of enzymes, such as trypsin or V8 protease, and the molecular weights of the digestion fragments can be used to search databases for sequences that match the molecular weights of the digestion fragments generated by the various enzymes. Alternatively, protein biomarkers can be sequenced using tandem MS technology. In this method, the protein is isolated by, for example, gel electrophoresis. A band containing the biomarker is cut out and the protein is subject to protease digestion. Individual protein fragments are separated by a first mass spectrometer. The fragment is then subjected to collision-induced cooling, which fragments the peptide and produces a polypeptide ladder. A polypeptide ladder is then analyzed by the second mass spectrometer of the tandem MS. The difference in masses of the members of the polypeptide ladder identifies the amino acids in the sequence. An entire protein can be sequenced this way, or a sequence fragment can be subjected to database mining to find identity candidates.

The preferred biological source for detection of the biomarkers is urine. However, in other embodiments, the biomarkers can be detected in serum.

The biomarkers of this invention are biomolecules. Accordingly, this invention provides these biomolecules in isolated form. The biomarkers can be isolated from biological fluids, such as urine or serum. They can be isolated by any method known in the art, based on both their mass and their binding characteristics. For example, a sample comprising the biomolecules can be subject to chromatographic fractionation, as described herein, and subject to further separation by, e.g., acrylamide gel electrophoresis. Knowledge of the identity of the biomarker also allows their isolation by immunoaffinity chromatography.

B. Modified Forms of Proteins as Biomarkers

It has been found that proteins frequently exist in a sample in a plurality of different forms characterized by detectably different masses. These forms can result from pre-translational modifications, post-translational modifications or both. Pre-translational modified forms include allelic variants, splice variants and RNA editing forms. Post-translationally modified forms include forms resulting from, among other things. proteolytic cleavage (e.g., fragments of a parent protein), glycosylation, phosphorylation, lipidation, oxidation, methylation, cystinylation, sulphonation and acetylation. The collection of proteins including a specific protein and all modified forms of it is referred to herein as a “protein cluster.” The collection of all modified forms of a specific protein, excluding the specific protein, itself, is referred to herein as a “modified protein cluster.” Modified forms of any biomarker of this invention also may be used, themselves, as biomarkers. In certain cases the modified forms may exhibit better discriminatory power in diagnosis than the specific forms set forth herein.

Modified forms of a biomarker can be initially detected by any methodology that can detect and distinguish the modified from the biomarker. A preferred method for initial detection involves first capturing the biomarker and modified forms of it, e.g., with biospecific capture reagents, and then detecting the captured proteins by mass spectrometry. More specifically, the proteins are captured using biospecific capture reagents, such as antibodies, interacting fusion proteins, aptamers or Affibodies that recognize the biomarker and modified forms of it. This method may also result in the capture of protein interactors that are bound to the proteins or that are otherwise recognized by antibodies and that, themselves, can be biomarkers. Preferably, the biospecific capture reagents are bound to a solid phase. Then, the captured proteins can be detected by SELDI mass spectrometry or by eluting the proteins from the capture reagent and detecting the eluted proteins by traditional MALDI or by SELDI. The use of mass spectrometry is especially attractive because it can distinguish and quantify modified forms of a protein based on mass and without the need for labeling.

Preferably, the biospecific capture reagent is bound to a solid phase, such as a bead, a plate, a membrane or a chip. Methods of coupling biomolecules, such as antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. For example, one can load multiple columns with derivatized beads, each column able to capture a single protein cluster. Alternatively, one can pack a single column with different beads derivatized with capture reagents against a variety of protein clusters, thereby capturing all the analytes in a single place. Accordingly, antibody-derivatized bead-based technologies can be used to detect the protein clusters. However, the biospecific capture reagents must be specifically directed toward the members of a cluster in order to differentiate them.

In yet another embodiment, the surfaces of biochips can be derivatized with the capture reagents directed against protein clusters either in the same location or in physically different addressable locations. One advantage of capturing different clusters in different addressable locations is that the analysis becomes simpler.

After identification of modified forms of a protein and correlation with the clinical parameter of interest, the modified form can be used as a biomarker in any of the methods of this invention. At this point, detection of the modified from can be accomplished by any specific detection methodology including affinity capture followed by mass spectrometry, or traditional immunoassay directed specifically to the modified form. Immunoassay requires biospecific capture reagents, such as antibodies, to capture the analytes. Furthermore, the assay must be designed to specifically distinguish a protein and modified forms of the protein. This can be done, for example, by employing a sandwich assay in which one antibody captures more than one form and second, distinctly labeled antibodies, specifically bind the various forms, thereby providing distinct detection of them. Antibodies can be produced by immunizing animals with the biomolecules. This invention contemplates traditional immunoassays including, for example, sandwich immunoassays including ELISA or fluorescence-based immunoassays, as well as other enzyme immunoassays.

### III. Detection of Biomarkers for Bladder Cancer

The biomarkers of this invention can be detected by any suitable method. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

Protein biochips are biochips adapted for the capture of polypeptides. Many protein biochips are described in the art. These include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828.

A. Detection by Mass Spectrometry

In a preferred embodiment, the biomarkers of this invention are detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In a further preferred method, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer.

1. SELDI

A preferred mass spectrometric technique for use in the invention is “Surface Enhanced Laser Desorption and Ionization” or “SELDI,” as described, for example, in U.S. Pat. Nos. 5,719,060 and No. 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent may be attached directly to the substrate of the selective surface, or the substrate may have a reactive surface that carries a reactive moiety that is capable of binding the capture reagent, e.g., through a reaction forming a covalent or coordinate covalent bond. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and H50 (hydrophobic); SAX-2, Q-10 and LSAX-30 (anion exchange); WCX-2, CM-10 and LWCX-30 (cation exchange); IMAC-3, IMAC-30 and IMAC 40 (metal chelate); and PS-10, PS-20 (reactive surface with carboimidizole, expoxide) and PG-20 (protein G coupled through carboimidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitriloacetic acid functionalities that adsorb transition metal ions, such as copper, nickel, zinc, and gallium, by chelation. Preactivated ProteinChip arrays have carboimidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); PCT International Publication No. WO 00/66265 (Rich et al., “Probes for a Gas Phase Ion Spectrometer,” Nov. 9, 2000); U.S. Pat. No. 6,555,813 (Beecher et al., “Sample Holder with Hydrophobic Coating for Gas Phase Mass Spectrometer,” Apr. 29, 2003); U.S. Patent Application No. U.S. 2003 0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application No. US 2003/0218130 A1 (Boschetti et al., “Biochips With Surfaces Coated With Polysaccharide-Based Hydrogels,” Apr. 14, 2003) and U.S. Patent Application No. 60/448,467, entitled “Photocrosslinked Hydrogel Surface Coatings” (Huang et al., filed Feb. 21, 2003).

In general, a probe with an adsorbent surface is contacted with the sample for a period of time sufficient to allow biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (HCA) and dihydroxybenzoic acid, ferulic acid, and hydroxyaceto-phenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 6,124,137 and PCT International Publication No. WO 03/64594 (Kitagawa, “Monomers And Polymers Having Energy Absorbing Moieties Of Use In Desorption/Ionization Of Analytes,” Aug. 7, 2003).

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker profile, pursuant to the present invention.

2. Other Mass Spectrometry Methods

In another mass spectrometry method, the biomarkers can be first captured on a chromatographic resin having chromatographic properties that bind the biomarkers. In the present example, this could include a variety of methods. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

3. Data Analysis

Analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. In Ciphergen's ProteinChip® software, data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, as part of Ciphergen's ProteinChip® software package, that can automate the detection of peaks. In general, this software functions by identifying signals having a signal-to-noise ratio above a selected threshold and labeling the mass of the peak at the centroid of the peak signal. In one useful application, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z cluster.

Software used to analyze the data can include code that applies an algorithm to the analysis of the signal to determine whether the signal represents a peak in a signal that corresponds to a biomarker according to the present invention. The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

4. General Protocol for SELDI Detection of Biomarkers for Bladder Cancer

A preferred protocol for the detection of the biomarkers of this invention is as follows. The biological sample to be tested, e.g., urine, preferably is subject to pre-fractionation before SELDI analysis. This simplifies the sample and improves sensitivity. A preferred method of pre-fractionation involves contacting the sample with an anion exchange chromatographic material, such as Q HyperD (BioSepra, SA). The bound materials are then subject to stepwise pH elution using buffers at pH 9, pH 7, pH 5 and pH 4. Various fractions containing the biomarker are collected.

The sample to be tested (preferably pre-fractionated) is then contacted with an affinity capture probe comprising a cation exchange adsorbent (preferably a WCX ProteinChip array (Ciphergen Biosystems, Inc.)) or an IMAC adsorbent (preferably an IMAC3 ProteinChip array (Ciphergen Biosystems, Inc.)), again as indicated in Table 2. The probe is washed with a buffer that will retain the biomarker while washing away unbound molecules. A suitable wash for each biomarker is the buffer identified in Table 2. The biomarkers are detected by laser desorption/ionization mass spectrometry.

Alternatively, if antibodies that recognize the biomarker are available, for example in the case of β2-microglobulin, cystatin, transferrin, transthyretin or albumin, these can be attached to the surface of a probe, such as a pre-activated PS10 or PS20 ProteinChip array (Ciphergen Biosystems, Inc.). These antibodies can capture the biomarkers from a sample onto the probe surface. Then the biomarkers can be detected by, e.g., laser desorption/ionization mass spectrometry.

B. Detection by Immunoassay

In another embodiment, the biomarkers of this invention can be measured by immunoassay. Immunoassay requires biospecific capture reagents, such as antibodies, to capture the biomarkers. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, sandwich immunoassays including ELISA or fluorescence-based immunoassays, as well as other enzyme immunoassays. In the SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### IV. Determination of Subject Bladder Cancer Status

A. Single Markers

The biomarkers of the invention can be used in diagnostic tests to assess bladder cancer status in a subject, e.g., to diagnose bladder cancer. The phrase “bladder cancer status” includes distinguishing, inter alia, bladder cancer v. non-bladder cancer and, in particular, bladder cancer v. non-bladder cancer normal or bladder cancer v. non-bladder cancer. Based on this status, further procedures may be indicated, including additional diagnostic tests or therapeutic procedures or regimens.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of actual positives that test as positive. Negative predictive value is the percentage of actual negatives that test as negative.

The biomarkers of this invention show a statistical difference in different bladder cancer statuses of at least p≦0.5, p≦0.05, p≦10⁻², p≦10⁻³, p≦10⁻⁴ or p≦10⁻⁵. Diagnostic tests that use these biomarkers alone or in combination show a sensitivity and specificity of at least 75%, at least 80%, at least 85%, at least 90%, at least 95%, at least 98% and about 100%.

Each biomarker listed in Table 1 is individually useful in aiding in the determination of bladder cancer status. The method involves, first, measuring the selected biomarker in a subject sample using the methods described herein, e.g., capture on a SELDI biochip followed by detection by mass spectrometry and, second, comparing the measurement with a diagnostic amount or cut-off that distinguishes a positive bladder cancer status from a negative bladder cancer status. The diagnostic amount represents a measured amount of a biomarker above which or below which a subject is classified as having a particular bladder cancer status. For example, if the biomarker is up-regulated compared to normal during bladder cancer, then a measured amount above the diagnostic cutoff provides a diagnosis of bladder cancer. Alternatively, if the biomarker is down-regulated during bladder cancer, then a measured amount below the diagnostic cutoff provides a diagnosis of bladder cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different bladder cancer statuses, as was done here, and drawing the cut-off to suit the diagnostician's desired levels of specificity and sensitivity.

B. Combinations of Markers

While individual biomarkers are useful diagnostic biomarkers, it has been found that a combination of biomarkers can provide, greater predictive value of a particular status than single biomarkers alone. Specifically, the detection of a plurality of biomarkers in a sample can increase the sensitivity and/or specificity of the test.

The protocols described in the Example below were used to generate mass spectra from 230 patient samples, 197 of which were diagnosed with bladder or other urogenital cancer and 33 of which did not exhibit any form of cancer. The peak masses and heights were abstracted into a discovery data set. This data set was used to train a learning algorithm employing classification and regression tree analysis (CART) (Ciphergen Biomarker Patterns Software™). In particular, CART chose many subsets of the peaks at random. For each subset, CART generated a best or near best decision tree to classify a sample as bladder cancer or non-bladder cancer. Among the many decision trees generated by CART, several had excellent sensitivity and specificity in distinguishing bladder cancer from non-bladder cancer.

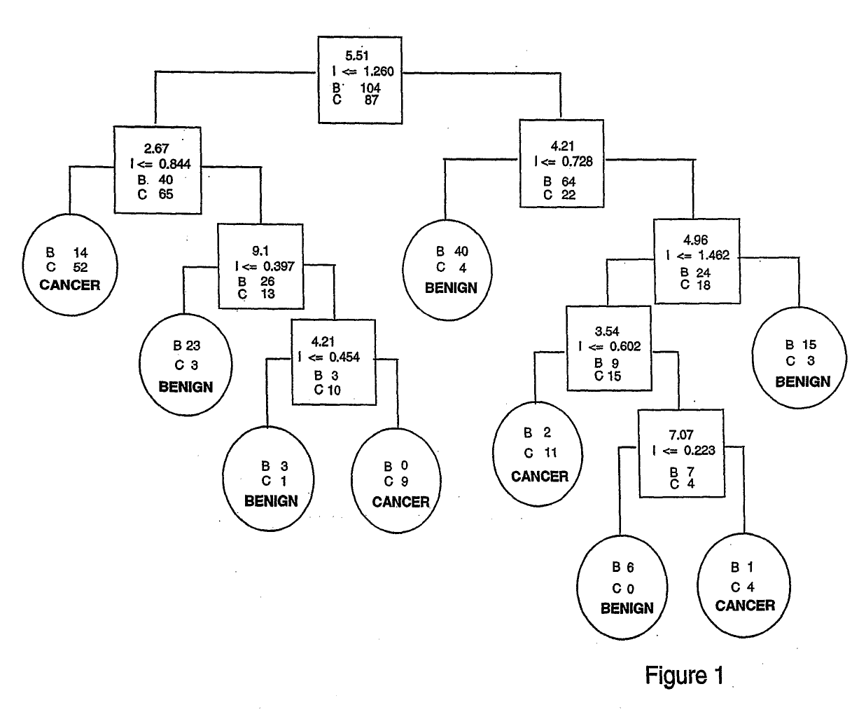

An exemplary decision tree is presented in FIG. 1. The tree uses biomarkers of mass to charge ratio M2670.00, M4210.00, M5510.00, and M9100.00 Da. Accordingly, these biomarkers are recognized as powerful classifiers for bladder cancer when used in combination with each other and, optionally, with other biomarkers. In particular, when used together or in further combination with M3540.00, M4960.00, and M7070.00 Da, these markers can distinguish bladder cancer from non-bladder cancer with sensitivities and specificities of at least 85%. Table 3 presents the performance of the decision tree presented in FIG. 1 in predicting bladder cancer. The number in parentheses denotes the number of correctly classified out of the total number of samples in the group. Sensitivity is defined as the ratio of detected cancers out of the total number of cancers included in the study. Specificity is defined as the percent of correctly identified control samples out of the total number of controls.

TABLE 3

| | Sensitivity (%) | Specificity (%) |
|---|---|---|
| Learning set | 87 (76/87) | 84 (87/104) |
| Cross-validation | 84 (73/87) | 74 (77/104) |
| Test set (SELDI) | 83 (15/18) | 67 (14/21) |
| Test set (BTAstat) | 78 (14/18) | 67 (14/21) |
| Test set (UBC) | 78 (14/18) | 67 (14/21) |

It is also noted that the specifics of the decision trees, in particular the cut-off values used in making branching decisions, depends on the details of the assay used to generate the discovery data set. The data acquisition parameters of the assay that produced the data used in the present analysis are provided in the Example. In developing a classification algorithm from, for example, a new sample set or a different assay protocol, the operator uses a protocol that detects these biomarkers and keys the learning algorithm to include them.

1. Decision Tree of FIG. 1

In one embodiment, biomarkers M2670.00, M4210.00, M5510.00, M9100.00, M3540.00, M4960.00, and M7070.00 are particularly useful in combination to classify bladder cancer v. non-bladder cancer. This combination is particularly useful in a recursive partitioning process as shown in FIG. 1. “C” represents bladder cancer patients and “B” represents “benign” patients (those that are non-bladder cancer normal or those that have benign or other non-bladder cancers). In one group, the presence of M5510.00 at a peak intensity threshold value of less than or equal to 1.260, and the presence of M2670.00 at a peak intensity of less than or equal to 0.844 may be correlated to a diagnosis of bladder cancer. In another group, the presence of M5510.00 at a peak intensity threshold value of less than or equal to 1.260, and the presence of M2670.00 at a peak intensity of greater than 0.844, and the presence of M9100.00 at a peak intensity of greater than 0.397, and the presence of M4210.00 at a peak intensity of greater than 0.454 may be correlated to a probable diagnosis of bladder cancer. In another group, the presence of M5510.00 at a peak intensity threshold value of greater than 1.260, and the presence of M4210.00 at a peak intensity of greater than 0.728, and the presence of M4960.00 at a threshold of less than or equal to 1.462, and the presence of M3540.00 at a peak intensity of less than or equal to 0.602 may be correlated to a probable diagnosis of bladder cancer. In another group, the presence of M5510.00 at a peak intensity threshold value of greater than 1.260, and the presence of M4210.00 at a peak intensity of greater than 0.728, and the presence of M4960.00 at a threshold of less than or equal to 1.462, and the presence of M3540.00 at a peak intensity of greater than 0.602, and the presence of M7070.00 at a threshold of greater than 0.223 may be correlated to a probable diagnosis of bladder cancer. In another group, the presence of M5510.00 at a peak intensity threshold value of less than or equal to 1.260, and the presence of M2670.00 at a peak intensity of greater than 0.844, and the presence of M9100.00 at a peak intensity of less than or equal to 0.397 may be correlated to a probable benign diagnosis. In another group, the presence of M5510.00 at a peak intensity threshold value of less than or equal to 1.260, and the presence of M2670.00 at a peak intensity of greater than 0.844, and the presence of M9100.00 at a peak intensity of greater than 0.397, and the presence of M4210.00 at a peak intensity of less than or equal to 0.454 may be correlated to a probable benign diagnosis. In another group, the presence of M5510.00 at a peak intensity threshold value of greater than 1.260, and the presence of M4210.00 at a peak intensity of less than or equal to 0.728 may be correlated to a probable benign diagnosis. In another group, the presence of M5510.00 at a peak intensity threshold value of greater than 1.260, and the presence of M4210.00 at a peak intensity of greater than 0.728, and the presence of M4960.00 at a threshold of greater than 1.462 may be correlated to a probable benign diagnosis. Finally, the presence of M5510.00 at a peak intensity threshold value of greater than 1.260, and the presence of M4210.00 at a peak intensity of greater than 0.728, and the presence of M4960.00 at a threshold of less than or equal to 1.462, and the presence of M3540.00 at a peak intensity of greater than 0.602, and the presence of M7070.00 at a threshold of less than or equal to 0.223 may be correlated to a probable benign diagnosis. Preferably, the combination of these groupings makes up a single classification tree for a diagnosis of bladder cancer. However, the present invention contemplates the use of these individual groupings alone or in combination with other groupings to aid in the diagnosis or identification of bladder cancer-positive and bladder cancer-negative patients. Thus, one or more of such groupings, preferably two or more, or more preferably, all of these groupings aid in the diagnosis.

2. SDS Algorithm

The same data set employed in the previously described CART analysis was used with the multi-stage Statistical Classification Strategy (SCS) (Institute for Biodiagnostics, National Research Council Canada, Winnipeg, MB Canada). SCS involves feature (mass peak) selection with a two-stage exhaustive search, using a wrapper approach. The classifier used in the wrapper was the simple linear discriminant with leave-one-out (LOO) crossvalidation. Once the optimally discriminatory peaks were found, the final classifier was obtained with a bootstrap-inspired approach.

The 7 best mass peaks identified by the SCS detected TCC in the test set with a sensitivity of 89% and a specificity of 81%. These seven peaks are at mass to charge ratios of M5400.00, M5830.00, M8490.00, M9420.00, M10800.00, M56500.00 and M67000.00 Da. When taking into account pairwise interactions among these seven peaks, the seven best of 35 possible linear and quadratic features consist of peaks M56500.00 and M67000.00 Da, the quadratic term of the M5830.00 Da peak and interaction between the M5400.00 Da & M8490.00 Da, the M5400.00 Da & M56500.00 Da, the M8490.00 Da & M67000.00 Da and the M9420.00 Da & M10800.00 Da peak pairs. These detected TCC in the test set with the same sensitivity of 89% as the seven best single peaks, but with an improved specificity of 91%. The SCS also identified a second set of 7 peaks at M3380.00, M5580.00, M5700.00, M5830.00, M9420.00, M17000.00 and M67000.00 Da. With this set of markers, the overall accuracy was higher on the test set, (87.2% vs. 84.6%), sensitivity reached 100.0%, and specificity was 76.2%. Accordingly, these biomarkers are recognized as powerful classifiers for bladder cancer when used in combination with each other and, optionally, with other biomarkers.

C. Subject Management

In certain embodiments of the methods of qualifying bladder cancer status, the methods further comprise managing subject treatment based on the status. Such management includes the actions of the physician or clinician subsequent to determining bladder cancer status. For example, if a physician makes a diagnosis of bladder cancer, then a certain regime of treatment, such as prescription or administration of chemotherapy or immunotherapy might follow. Alternatively, a diagnosis of non-bladder cancer or non-bladder cancer might be followed with further testing to determine a specific disease that might the patient might be suffering from. Also, if the diagnostic test gives an inconclusive result on bladder cancer status, further tests may be called for.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the presence or absence in a test subject of any the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### V. Generation of Classification Algorithms for Qualifying Bladder Cancer Status

In some embodiments, data derived from the spectra (e.g., mass spectra or time-of-flight spectra) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART-classification and regression trees), artificial neural networks such as back propagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for bladder cancer. The classification algorithms, in turn, form the base for diagnostic tests by providing diagnostic values (e.g., cut-off points) for biomarkers used singly or in combination.

### VI. Kits for Detection of Biomarkers for Bladder Cancer

In another aspect, the present invention provides kits for qualifying bladder cancer status, which kits are used to detect biomarkers according to the invention. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI such as ProteinChip® arrays. In the case of biospecific capture reagents, the kit can comprise a solid support with a reactive surface, and a container comprising the biospecific capture reagent.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular biomarkers to be detected.

In yet another embodiment, the kit can comprise one or more containers with biomarker samples, to be used as standard(s) for calibration.

### VII. Use of Biomarkers for Bladder Cancer in Screening Assays

The methods of the present invention have other applications as well. For example, the biomarkers can be used to screen for compounds that modulate the expression of the biomarkers in vitro or in vivo, which compounds in turn may be useful in treating or preventing bladder cancer in patients. In another example, the biomarkers can be used to monitor the response to treatments for bladder cancer. In yet another example, the biomarkers can be used in heredity studies to determine if the subject is at risk for developing bladder cancer.

Thus, for example, the kits of this invention could include a solid substrate having a hydrophobic function, such as a protein biochip (e.g., a Ciphergen H50 ProteinChip array, e.g., ProteinChip array) and a sodium acetate buffer for washing the substrate, as well as instructions providing a protocol to measure the biomarkers of this invention on the chip and to use these measurements to diagnose bladder cancer.

Compounds suitable for therapeutic testing may be screened initially by identifying compounds which interact with one or more biomarkers listed in Table 1. By way of example, screening might include recombinantly expressing a biomarker listed in Table 1, purifying the biomarker, and affixing the biomarker to a substrate. Test compounds would then be contacted with the substrate, typically in aqueous conditions, and interactions between the test compound and the biomarker are measured, for example, by measuring elution rates as a function of salt concentration. Certain proteins may recognize and cleave one or more biomarkers of Table 1, in which case the proteins may be detected by monitoring the digestion of one or more biomarkers in a standard assay, e.g., by gel electrophoresis of the proteins.

In a related embodiment, the ability of a test compound to inhibit the activity of one or more of the biomarkers of Table 1 may be measured. One of skill in the art will recognize that the techniques used to measure the activity of a particular biomarker will vary depending on the function and properties of the biomarker. For example, an enzymatic activity of a biomarker may be assayed provided that an appropriate substrate is available and provided that the concentration of the substrate or the appearance of the reaction product is readily measurable. The ability of potentially therapeutic test compounds to inhibit or enhance the activity of a given biomarker may be determined by measuring the rates of catalysis in the presence or absence of the test compounds. The ability of a test compound to interfere with a non-enzymatic (e.g., structural) function or activity of one of the biomarkers of Table 1 may also be measured. For example, the self-assembly of a multi-protein complex which includes one of the biomarkers of Table 1 may be monitored by spectroscopy in the presence or absence of a test compound. Alternatively, if the biomarker is a non-enzymatic enhancer of transcription, test compounds which interfere with the ability of the biomarker to enhance transcription may be identified by measuring the levels of biomarker-dependent transcription in vivo or in vitro in the presence and absence of the test compound.

Test compounds capable of modulating the activity of any of the biomarkers of Table 1 may be administered to patients who are suffering from or are at risk of developing bladder cancer or other cancer. For example, the administration of a test compound which increases the activity of a particular biomarker may decrease the risk of bladder cancer in a patient if the activity of the particular biomarker in vivo prevents the accumulation of proteins for bladder cancer. Conversely, the administration of a test compound which decreases the activity of a particular biomarker may decrease the risk of bladder cancer in a patient if the increased activity of the biomarker is responsible, at least in part, for the onset of bladder cancer.

In an additional aspect, the invention provides a method for identifying compounds useful for the treatment of disorders such as bladder cancer which are associated with increased levels of modified forms of any of the biomarkers of Table 1. For example, in one embodiment, cell extracts or expression libraries may be screened for compounds which catalyze the cleavage of a full-length biomarker to form a truncated form of the biomarker. In one embodiment of such a screening assay, cleavage of a biomarker may be detected by attaching a fluorophore to the biomarker which remains quenched when the biomarker is uncleaved but which fluoresces when the protein is cleaved. Alternatively, a version of full-length biomarker modified so as to render the amide bond between amino acids x and y uncleavable may be used to selectively bind or “trap” the cellular protease which cleaves a full-length biomarker at that site in vivo. Methods for screening and identifying proteases and their targets are well-documented in the scientific literature, e.g., in Lopez-Ottin et al. (Nature Reviews, 3:509-519 (2002)).

In yet another embodiment, the invention provides a method for treating or reducing the progression or likelihood of a disease, e.g., bladder cancer, which is associated with the increased levels of truncated forms of any of the biomarkers of Table 1. For example, after one or more proteins have been identified which cleave a full-length biomarker, combinatorial libraries may be screened for compounds which inhibit the cleavage activity of the identified proteins. Methods of screening chemical libraries for such compounds are well-known in art. See, e.g., Lopez-Otin et al. (2002). Alternatively, inhibitory compounds may be intelligently designed based on the structure of any of the biomarkers of Table 1.

Compounds which impart a truncated biomarker with the functionality of a full-length biomarker are likely to be useful in treating conditions, such as bladder cancer, which are associated with the truncated form of the biomarker. Therefore, in a further embodiment, the invention provides methods for identifying compounds which increase the affinity of a truncated form of any of the biomarkers of Table 1 for its target proteases. For example, compounds may be screened for their ability to impart a truncated biomarker with the protease inhibitory activity of the full-length biomarker. Test compounds capable of modulating the inhibitory activity of a biomarker or the activity of molecules which interact with a biomarker may then be tested in vivo for their ability to slow or stop the progression of bladder cancer in a subject.

At the clinical level, screening a test compound includes obtaining samples from test subjects before and after the subjects have been exposed to a test compound. The levels in the samples of one or more of the biomarkers listed in Table 1 may be measured and analyzed to determine whether the levels of the biomarkers change after exposure to a test compound. The samples may be analyzed by mass spectrometry, as described herein, or the samples may be analyzed by any appropriate means known to one of skill in the art. For example, the levels of one or more of the biomarkers listed in Table 1 may be measured directly by Western blot using radio- or fluorescently-labeled antibodies which specifically bind to the biomarkers. Alternatively, changes in the levels of mRNA encoding the one or more biomarkers may be measured and correlated with the administration of a given test compound to a subject. In a further embodiment, the changes in the level of expression of one or more of the biomarkers may be measured using in vitro methods and materials. For example, human tissue cultured cells which express, or are capable of expressing, one or more of the biomarkers of Table 1 may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with bladder cancer, test compounds will be screened for their ability to slow or stop the progression of the disease.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results.

### VII. Examples

### Example 1

### Discovery of Biomarkers for Bladder Cancer Using CART Analysis

Urine Samples

Urine samples were obtained patients seen in the Departments of Urology at the Eastern Virginia Medical School in Norfolk, Va. and Laikon Hospital in Athens, Greece. Bladder cancer samples (n=105) were obtained from patients aged 27-91 years, with a mean age of 71.3. Non-bladder cancer samples (n=125) were obtained from patients aged 34-86 years, with a mean age of 62.6. In all cases, patients were consented according to the regulations for human subject protection of each institution. The urine samples were aliquoted and frozen at −80° C. until thawed specifically for SELDI analysis.

Healthy controls (n=33) included volunteers with no evidence of disease, and healthy individuals (i.e., no history or evidence of urologic cancer) participating in the prostate and screening program at EVMS. Bladder cancer (n=105 patients) was histologically or cytologically confirmed at the time of specimen collection and the vast majority of cases involved newly diagnosed cancers (n=83). In the case of recurrences (n=22) none of the patients had received chemo- or immunotherapy within 3 months prior to specimen collection. Other urogenital diseases (n=92) included clinical or pathologically confirmed benign prostatic hyperplasia (BPH) (n=47), urinary tract infections (n=13), urolethiasis (13), amyloidosis (n=1), prostate cancer (n=11), renal cell carcinoma (5), and seminoma (1).

Grading was assessed using the World Health Organization (WHO) system. Tumor stage and grade of patients with TCC are shown in Table 4 below.

TABLE 4

| | No. of | No. of | | No. of | No. of |
|---|---|---|---|---|---|
| | samples | samples | | samples | samples |
| Stage | (L) | (T) | Grade | (L) | (T) |
| Ta | 36 | 9 | I | 5 | 3 |
| T1 | 25 | 1 | II | 31 | 3 |
| T2 | 18 | 4 | III | 51 | 12 |
| T3-T4 | 2 | 0 | | | |
| Ta CIS | 3 | 1 | | | |
| T1 CIS | 2 | 2 | | | |
| T2 CIS | 1 | 0 | | | |

SELDI Processing of Urine Samples

Prior to their application on protein chips, urine samples were briefly centrifuged (1 min, 10,000 rpm) for the removal of exfoliated cells. The supernatants were then applied to the chips using a Coulter Beckman Biomek 2000 Laboratory Automation Workstation as follows: 63 μl of urine were mixed with 21 μl of 9M urea-2% CHAPS-50 mM Tris pH 9 buffer for 30 minutes at 4° C., followed by the addition of 84 μl of binding buffer (100 mM sodium acetate, pH 4.0). One hundred microliters of the diluted samples were then applied onto the weak cation exchange (WCX) chips using a bioprocessor (Ciphergen Biosystems Inc.). Following a 45-minute incubation, non-specifically bound molecules were removed by three brief washes in 200 μl binding buffer followed by three washes with 200 μl HPLC-gradient water. Sinapinic acid (2X1 μl of 50% SPA in 50% ACN-0.1% TFA) was applied to the chip array surface and mass spectrometry performed using a PBS2 SELDI-TOF mass spectrometer (Ciphergen Biosystems Inc.). Mass spectral data were collected by averaging the output from a total of 192 laser shots at a laser intensity of 220. Mass calibration was performed using the all-in-one peptide standard (Ciphergen Biosystems Inc.) and specifically Vasopressin (1084.25), Somatostatin (1637.9), Insulin B-chain (bovine; 3495.94), Insulin (human recombinant; 5807.65), and Hirudin (7033.61). All samples were processed in duplicate.

One urine sample designated as quality control (QC) was included in every chip array to estimate reproducibility of the profiling assay. Three randomly selected peaks with masses at 2.8, 4.8, and 11.8 kDa were utilized to estimate the mass location and peak intensity coefficients of variations (CV). From the analysis of a total of 89 QC spectra, the mass CV was found to be 0.05-0.3% and the intensity CV 40-70%.

Before analysis, the data was divided into two sets: a training set consisting of 191 samples (87 bladder cancer, 73 other urogenital diseases, and 31 normal), and a test set of 39 samples (18 bladder cancer, 19 other urogenital diseases, and 2 normal).

Processing of SELDI Data

Spectral peaks were labeled and their intensities normalized to the total ion current (mass range 2.5-30 kDa) to account for variation in ionization efficiencies, using the SELDI software (Version 3.1). Peak masses were aligned and clustering was performed using the Biomarker Wizard software (Ciphergen Biosystems). Specifically the settings for peak labeling and alignment were the following: in the 2.5 to 30 kDa mass range: signal/noise (first pass)=3; minimum peak threshold=10%; cluster mass window=0.3%; and signal/noise (second pass)=1.5. In the 20 to 100 kDa range: signal/noise (first pass)=5; minimum peak threshold=10%; mass error=1% and signal/noise (second pass)=2.5. With these settings a total of 101 peaks per spectrum were detected (90 in the 2.5-30 kDa and 11 in the 30-150K mass range). Intensity values for each of these peaks were exported to an Excel file, and averaged for each duplicate spectra.

Pattern recognition and sample classification were performed using the Biomarker Pattern Software (Ciphergen Biosystems Inc.). The decision tree was generated using the Gini method non-linear combinations. Construction of the decision tree classification algorithm was performed as described by Breiman, L., et al., Classification and Regression Trees, (1984). Details regarding the Classification and Regression Tree (CART) and the artificial intelligence bioinformatics algorithm incorporated within the BioMarker Patterns software program have also been described in Bertone, P., et al., Nucleic Acids Res. 29: 2884-2898 (2001); Kosuda, S., et al., Ann. Nucl. Med. 16: 263-271 (2002).

Briefly, classification trees split the data into two bins based on decision rules (squares, FIG. 1). The rules are formed by the peak intensities being either greater or lesser than a specific value for each selected mass. Samples that follow the rule (i.e. peak intensity is equal to or less than the cut-off intensity value) go to the left daughter node; others go to the right daughter node. When splitting can no longer be performed, terminal nodes are generated and classified according to the samples in the majority; in this case the terminal node is either classified as cancer or benign/normal (circles, FIG. 1).

A 10-fold cross validation analysis was performed as an initial evaluation of the test error of the algorithm. Briefly, this process involves splitting up the data set iteratively into 10 random segments and using nine of them for training and the tenth as a test set for the algorithm. Multiple trees were initially generated, by varying the splitting factor by increments of 0.1. The peaks that formed the main splitters of the tree with the highest prediction rates in the cross-validation analysis were then selected and the tree was rebuilt based on these peaks alone. This tree was then challenged to classify the samples included in the test set. The classification provided by the algorithm was compared to the specimen pathologic diagnosis.

UBC and BTAstat Test Analysis of Urine Samples

The UBC (IDL Biotech, Sollentona, Sweden) and BTAstat (Bion Diagnostic Sciences, Redmond, Wash.) tests were performed according to the manufacturer's instructions. For UBC, a cut-off value of 12 μg/l was selected based on receiver operating characteristics curve analysis (Giannopoulos et al., J. Urol. 166(2): 488-9 (2001)).

CART Analysis

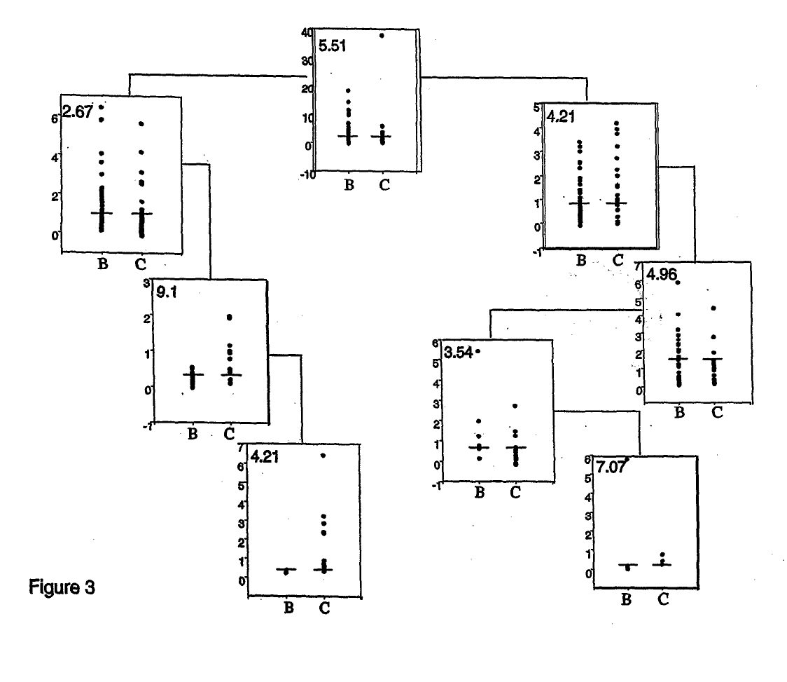

The benign, other cancers and normal samples were pooled to form the control group. Seven protein peaks at M670.00, M4210.00, M5510.00, M9100.00, M3540.00, M4960.00, and M7070.00 Da generated a decision tree that provided optimal discrimination between the bladder cancer and control group during the algorithm evaluation (FIG. 1). The sample segregation in the decision nodes, as well as the samples' intensity levels for the main splitter, are shown in FIG. 3. Representative mass spectra of the splitters are shown on FIG. 2. Peak intensities between different groups were compared with student's t-test. With the exception of the M7070.00 and M9100.00 Da peaks, the rest of the main splitters had significantly different intensity levels between the cancer and control groups (Table 5).

TABLE 5

| Splitter (Da) | p |
|---|---|
| M2670.00 | 0.006 |
| M3540.00 | 0.007 |
| M4210.00 | 0.042 |
| M4960.00 | <0.001 |
| M5510.00 | 0.044 |
| M7070.00 | 0.41 |
| M9100.00 | 0.25 |

In the cross-validation analysis, the decision tree predicted bladder cancer with 78.5% (150/191) accuracy (Table 3). In the blinded test set, this tree classified accurately 74% (29/39) of the samples. By comparison, in the same set of samples, the BTAstat and UBC tests predicted bladder cancer with 72% (28/39) accuracy (Table 3). Interestingly, the SELDI decision tree detected 5 out of 6 of the low grade (I and II) tumors while the BTAstat detected 2 out of 6 and the UBC test found 4 out of 6. Nevertheless, the responses of the three tests were found to be independent of each other (P>0.05) and therefore their combination did not improve the overall diagnostic rates.

A summation of the classification results from the decision tree is presented for the training and test sets in Table 6 below.

TABLE 6
Decision Tree Classification of the Bladder
Cancer Training and Test Sets

| | | | |
|---|---|---|---|
| | Normal and other | Bladder | Misclassified |
| Sample | urogenital diseases | cancer | Rate |

A. Training Set

| | | | | | | |
|---|---|---|---|---|---|---|
| Normal and other | 87 | 83.7% | 17 | 16.3% | 17 | 16.3% |
| urogenital diseases | | | | | | |
| (N = 104) | | | | | | |
| Bladder cancer | 11 | 12.6% | 76 | 87.4% | 11 | 12.6% |
| (N = 87) | | | | | | |

| | | |
|---|---|---|
| Total Samples | 28 | 14.7% |
| (N = 191) | | |

B. Test Set

| | | | | | | |
|---|---|---|---|---|---|---|
| Normal and other | 14 | 66.7% | 7 | 33.3% | 7 | 33.3% |
| urogenital diseases | | | | | | |
| (N = 21) | | | | | | |
| Bladder cancer | 3 | 16.7% | 15 | 83.3% | 3 | 16.7% |
| (N = 18) | | | | | | |

| | | |
|---|---|---|
| Total Samples | 10 | 25.6% |
| (N = 39) | | |

### Example 2

### Discovery of Biomarkers for Bladder Cancer Using SCS Analysis

The 89-peak SELDI dataset as described in Example 1 was analyzed with SCS. The SCS strategy was developed to deal specifically with the analysis of biomedical data, characterized by typically large (ο(1000-10000)) number of features (e.g. m/z values) and few (ο(10-100)) samples. The SCS is a multi-stage approach. Before the first, feature reduction stage, data transformations are usually applied (for spectra, these can be scaling to unit area, “whitening”, smoothing, peak alignment, replacing the spectra by their first or second derivatives, etc). Both the original data and its rank-ordered version were used. (Rank ordering is a nonlinear transformation that replaces in each spectrum the actual peak intensity values by their ranks.) This helps reduce the influence of accidentally large or small feature values. Feature (peak) selection was then applied to both original and rank-ordered data.

Exhaustive search (ES) for the best 7 out of 89 features is computationally not feasible. However, finding the best 5 out of 89 is. Using a frequency count of how many times one of the 89 peaks appeared in the best solutions, 30 peaks were selected. Best 7 of 30 is quite feasible, even if a wrapper approach is used, i.e. if classification accuracy is used as the criterion for selecting the features. For the classifier of this wrapper approach, LDA was used with leave-one-out crossvalidation. Of the 2,035,800 possible 7-peak feature sets, the best 6 sets that maintained an acceptable balance between sensitivity (false positive, FP rate) and specificity (false negative, FN rate) were selected as candidates. Candidate selection was performed by minimizing the difference between (FP-FN)² at the feature selection stage and also imposing a larger penalty for misclassifying the TCC samples

Each of the 6 feature sets was used to develop the best corresponding classifier. Inspired by the “resampling with replacement” philosophy of Efron's bootstrap approach (Efron, B. and Gong, G., American Statistician 37(1): 3648 (1983)), a robust classifier was created by randomly selecting about half of the samples (in a stratified manner) as a training set, developing a crossvalidated classifier, and using the other half to test classifier efficacy. The training samples are then returned to the original pool and the process repeated, usually B=5,000-10,000 times. The optimized classifier coefficients for all B random splits are saved. The improvement over conventional approaches is that the final classifier is produced as the weighted average of these B sets of coefficients. The weight for classifier j is Qⱼ=κⱼCⱼ^{1/2}, with 0≦Cⱼ≦1 the fraction of crisp (p≧0.75) class assignment probabilities, and κⱼ is Cohen's chance-corrected measure of agreement [16], ˜0≦κⱼ≦1; κⱼ=1 signifies perfect classification. The B Qⱼ values found for the less optimistic test sets were used (Somodjai, et al., A Data-Driven, Flexible Machine Learning Strategy for the Classification of Biomedical Data, Chapter in “Artificial Intelligence Methods for Systems Biology”, Dubetzky, W. and Azuaje, F. (eds.), Kluwer Academic Publ. (in press)). For these studies, using the top test Qⱼ gave the best classifier.

Discussion

Using SELDI/TOF-MS techniques coupled with application of bioinformatic tools, the decision tree achieved 83-87% specificity/67% sensitivity and SCS achieved 89% specificity/81% sensitivity for detection of bladder cancer in a rapid and reproducible manner and in a large number of samples. While not intending to be bound by a particular theory, it appears that the protein pattern, rather than individual protein alteration, may be more important for differentiating normal healthy individuals from those who have, or are likely to develop, bladder cancer. The high sensitivities and specificities achieved in these studies using SELDI/TOF-MS techniques, coupled with robust artificial intelligence classification algorithms, identified protein patterns in urine samples that distinguished non-bladder cancer controls from bladder cancer patients. These techniques provide data that are easy to accumulate and should lend itself readily to clinical use.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. In addition, all references and patents cited herein are indicative of the level of skill in the art and hereby incorporated by reference in their entirety.

What is claimed is:

1. A method for qualifying bladder cancer status in a subject comprising:
a) measuring at least one biomarker in a biological sample from the subject, wherein the at least one biomarker is selected from the group consisting of Marker 1, Marker 2, Marker 3, Marker 4, Marker 5, Marker 6, Marker 7, Marker 8, Marker 9, Marker 10, Marker 11, and Marker 12; and
b) correlating the measurement with bladder cancer status.

2. The method of claim 1, comprising measuring a plurality of said biomarkers.

3. The method of claim 2, wherein the plurality comprises at least 3 biomarkers.

4. The method of claim 2, wherein the plurality comprises at least 4 biomarkers.

5. The method of claim 1, further comprising measuring Marker 13.

6. The method of claim 1, further comprising measuring Marker 14.

7. The method of claim 1, further comprising measuring Marker 15.

8. The method of claim 1, further comprising measuring Marker 16.

9. The method of claim 1, further comprising measuring Marker 17.

10. The method of claim 1, further comprising measuring Marker 18.

11. A method for qualifying bladder cancer status in a subject comprising:
a) measuring a plurality of biomarkers in a biological sample from the subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and
b) correlating the measurement with bladder cancer status.

12. The method of any of claims 1 or 11, wherein the at least one biomarker is measured by capturing the biomarker on an adsorbent surface of a SELDI probe and detecting the captured biomarkers by laser desorption-ionization mass spectrometry.

13. The method of any of claims 1 or 11, wherein the at least one biomarker is measured by immunoassay.

14. The method of any of claims 1 or 11, wherein the sample is urine.

15. The method of any of claims 1 or 11, wherein the sample is serum.

16. The method of any of claims 1 or 11, wherein the correlating is performed by a software classification algorithm.

17. The method of any of claims 1 or 11, wherein bladder cancer status is selected from bladder cancer and non-bladder cancer.

18. The method of any of claims 1 or 11, further comprising (c) managing subject treatment based on the status.

19. The method of claim 12, wherein the adsorbent is a cation exchange adsorbent.

20. The method of claim 12, wherein the adsorbent is a biospecific adsorbent.

21. The method of claim 18, further comprising (d) measuring the at least one biomarker after subject management.

22. A method comprising measuring at least one biomarker in a sample from a subject, wherein the at least one biomarker is selected from the group consisting of Marker 1, Marker 2, Marker 3, Marker 4, Marker 5, Marker 6, Marker 7, Marker 8, Marker 9, Marker 10, Marker 11, and Marker 12.

23. The method of claim 22, comprising measuring a plurality of said biomarkers.

24. The method of claim 23, wherein the plurality comprises at least 3 biomarkers.

25. The method of claim 23, wherein the plurality comprises at least 4 biomarkers.

26. The method of claim 22, further comprising measuring Marker 13.

27. The method of claim 22, further comprising measuring Marker 14.

28. The method of claim 22, further comprising measuring Marker 15.

29. The method of claim 22, further comprising measuring Marker 16.

30. The method of claim 22, further comprising measuring Marker 17.

31. The method of claim 22, further comprising measuring Marker 18.

32. A method comprising measuring a plurality of biomarkers in a sample from a subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18.

33. The method of any of claims 22 or 32, wherein the biomarker is measured by capturing the biomarker on an adsorbent surface of a SELDI probe and detecting the captured biomarkers by laser desorption-ionization mass spectrometry.

34. The method of any of claims 22 or 32, wherein the sample is urine.

35. The method of any of claims 22 or 32, wherein the sample is serum.

36. The method of claim 33, wherein the adsorbent is a cation exchange adsorbent.

37. The method of claim 33, wherein the adsorbent is a biospecific adsorbent.

38. A kit comprising:
a) a solid support comprising at least one capture reagent attached thereto, wherein the capture reagent binds at least one biomarker selected from the group consisting of Marker 1, Marker 2, Marker 3, Marker 4, Marker 5, Marker 6, Marker 7, Marker 8, Marker 9, Marker 10, Marker 11, and Marker 12; and
b) instructions for using the solid support to detect the at least one biomarker.

39. The kit of claim 38, comprising instructions for using the solid support to detect a plurality of said biomarkers.

40. The kit of claim 39, wherein the plurality comprises at least 3 biomarkers.

41. The kit of claim 39, wherein the plurality comprises at least 4 biomarkers.

42. The kit of claim 38, further comprising instructions for using the solid support to Marker 13.

43. The kit of claim 38, further comprising instructions for using the solid support to detect Marker 14.

44. The kit of claim 38, further comprising instructions for using the solid support to detect Marker 15.

45. The kit of claim 38, further comprising instructions for using the solid support to detect Marker 16.

46. The kit of claim 38, further comprising instructions for using the solid support to detect Marker 17.

47. The kit of claim 38, further comprising instructions for using the solid support to detect Marker 18.

48. A kit comprising:
a) a solid support comprising at least one capture reagent attached thereto, wherein the capture reagent binds a plurality of biomarkers, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and
b) instructions for using the solid support to detect the plurality of biomarkers.

49. The kit of any of claims 38 or 48, wherein the solid support comprising a capture reagent is a SELDI probe.

50. The kit of any of claims 38 or 48, additionally comprising (c) an anion exchange chromatography adsorbent.

51. The kit of claim 49, wherein the capture reagent is a cation exchange adsorbent.

52. A kit comprising:
a) a solid support comprising at least one capture reagent attached thereto, wherein the capture reagents bind at least one biomarker selected from the group consisting of Marker 1, Marker 2, Marker 3, Marker 4, Marker 5, Marker 6, Marker 7, Marker 8, Marker 9, Marker 10, Marker 11, and Marker 12; and
b) a container containing at least one of the biomarkers.

53. The kit of claim 52, wherein the container comprises a plurality of said biomarkers.

54. The kit of claim 53, wherein the plurality comprises at least 3 biomarkers.

55. The kit of claim 53, wherein the plurality comprises at least 4 biomarkers.

56. The kit of claim 52, further comprising instructions for using the solid support to detect Marker 13.

57. The kit of claim 52, further comprising instructions for using the solid support to detect Marker 14.

58. The kit of claim 52, further comprising instructions for using the solid support to detect Marker 15.

59. The kit of claim 52, further comprising instructions for using the solid support to detect Marker 16.

60. The kit of claim 52, further comprising instructions for using the solid support to detect Marker 17.

61. The kit of claim 52, further comprising instructions for using the solid support to detect Marker 18.

62. A kit comprising:
a) a solid support comprising at least one capture reagent attached thereto, wherein the capture reagents bind a plurality of biomarkers, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and
b) a container containing at least one of the biomarkers.

63. The kit of any of claims 52 or 62, wherein the solid support comprising a capture reagent is a SELDI probe.

64. The kit of any of claims 52 or 62, additionally comprising (c) an anion exchange chromatography adsorbent.

65. The kit of claim 63, wherein the capture reagent is a cation exchange adsorbent.

66. A software product comprising:
a) code that accesses data attributed to a sample, the data comprising measurement of at least one biomarker in the sample, the biomarker selected from the group consisting of Marker 1, Marker 2, Marker 3, Marker 4, Marker 5, Marker 6, Marker 7, Marker 8, Marker 9, Marker 10, Marker 11, and Marker 12; and
b) code that executes a classification algorithm that classifies the bladder cancer status of the sample as a function of the measurement.

67. The software product of claim 66, wherein the classification algorithm classifies the bladder cancer status of the sample as a function of the measurement of a plurality of said biomarkers.

68. The software product of claim 67, wherein the plurality comprises at least 3 biomarkers.

69. The software product of claim 67, wherein the plurality comprises at least 4 biomarkers.

70. The software product of claim 66, wherein the classification algorithm classifies the bladder cancer status of the sample further as a function of the measurement of Marker 13.

71. The software product of claim 66, wherein the classification algorithm classifies the bladder cancer status of the sample further as a function of the measurement of Marker 14.

72. The software product of claim 66, wherein the classification algorithm classifies the bladder cancer status of the sample further as a function of the measurement of Marker 15.

73. The software product of claim 66, wherein the classification algorithm classifies the bladder cancer status of the sample further as a function of the measurement of Marker 16.

74. The software product of claim 66, wherein the classification algorithm classifies the bladder cancer status of the sample further as a function of the measurement of Marker 17.

75. The software product of claim 66, wherein the classification algorithm classifies the bladder cancer status of the sample further as a function of the measurement of Marker 18.

76. A software product comprising:
a) code that accesses data attributed to a sample, the data comprising measurement of a plurality of biomarkers in the sample, and wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18; and
b) code that executes a classification algorithm that classifies the bladder cancer status of the sample as a function of the measurement.

77. A purified biomolecule selected from the group of biomarkers consisting of Marker 1, Marker 2, Marker 3, Marker 4, Marker 5, Marker 6, Marker 7, Marker 8, Marker 9, Marker 10, Marker 11, and Marker 12.

78. A method comprising detecting a biomarker selected from the group consisting of Marker 1, Marker 2, Marker 3, Marker 4, Marker 5, Marker 6, Marker 7, Marker 8, Marker 9, Marker 10, Marker 11, and Marker 12 by mass spectrometry or immunoassay.

79. A method comprising detecting a plurality of biomarkers by mass spectrometry or immunoassay, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18.

80. A method comprising communicating to a subject a diagnosis relating to bladder cancer status determined from the correlation of biomarkers in a sample from the subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12.

81. A method comprising communicating to a subject a diagnosis relating to bladder cancer status determined from the correlation of a plurality of biomarkers in a sample from the subject, wherein at least one biomarker is selected from the group consisting of Marker 1, Marker 2, Marker 3, Marker 4, Marker 5, Marker 6, Marker 7, Marker 8, Marker 9, Marker 10, Marker 11, and Marker 12 and at least one biomarker is selected from the group consisting of Marker 13, Marker 14, Marker 15, Marker 16, Marker 17, and Marker 18.

82. The method of any of claims 80 or 81, wherein the diagnosis is communicated to the subject via a computer-generated medium.

83. A method for identifying a compound that interacts with any of the biomarkers selected from the group consisting of Marker 1, Marker 2, Marker 3, Marker 4, Marker 5, Marker 6, Marker 7, Marker 8, Marker 9, Marker 10, Marker 11, and Marker 12, wherein said method comprises:
a) contacting the biomarker with a test compound; and
b) determining whether the test compound interacts with the biomarker.

84. A method for modulating the concentration of a biomarker selected from the group consisting of Marker 1, Marker 2, Marker 3, Marker 4, Marker 5, Marker 6, Marker 7, Marker 8, Marker 9, Marker 10, Marker 11, Marker 12, Marker 13, Marker 14, Marker 15, Marker 16, Marker 17, and Marker 18 in a cell, wherein said method comprises:
a) contacting said cell with a protease inhibitor, wherein said protease inhibitor prevents cleavage of said biomarker.

85. A method of treating a condition in a subject, wherein said method comprises administering to a subject a therapeutically effective amount of a compound which modulates the expression or activity of a protease which cleaves a biomarker selected from the group consisting of Marker 1, Marker 2, Marker 3, Marker 4, Marker 5, Marker 6, Marker 7, Marker 8, Marker 9, Marker 10, Marker 11, Marker 12, Marker 13, Marker 14, Marker 15, Marker 16, Marker 17, and Marker 18.

86. The method of claim 85, wherein said condition is bladder cancer.
